(19) United States
(12) Patent Application Publication
(10) Pub. No.: US 2018/0169142 A1
(43) Pub. Date: Jun. 21, 2018

(54) METHODS, MEANS AND COMPOSITIONS FOR IMPROVING OUTCOMES OF SURGICAL INTERVENTIONS AND INFLAMMATORY SEQUEL

(71) Applicant: Nobilis Therapeutics, Inc., Portland, OH (US)

(72) Inventors: Thomas ICHIM, San Diego, CA (US); Vlad BOGIN, Portland, OR (US)

(73) Assignee: NOBILIS THERAPEUTICS, INC., Portland, OR (US)

(21) Appl. No.: 15/847,806

(22) Filed: Dec. 19, 2017

Related U.S. Application Data
(60) Provisional application No. 62/436,408, filed on Dec. 19, 2016.

(51) Int. Cl. A61K 33/00

(52) U.S. Cl. CPC A61K 33/00

(57) ABSTRACT

Disclosed are methods, means and compositions of matter useful for improving outcomes after surgical interventions, said improvements including reduction of pain, acceleration of healing, reduction of fibrosis, inflammation and formation of surgical adhesions. Said surgical interventions include manipulation of tissues by means of mechanical, surgical, and ablative means. Said compositions include noble gases which directly or indirectly induce antifibrotic, anti-inflammatory and pain ameliorating effects. Said gases are injected directly during the procedure of tissue manipulation, or contacted with fluids, or fluids containing cells that are subsequently injected adjacent to said tissue being manipulated. In one embodiment of the invention, laparoscopic surgery is performed using noble gases to insufflate the said body cavities. In another embodiment, said noble gases are used to treat liquids added during said manipulations of tissue, including surgical manipulation. In a specific embodiment, the invention teaches the use of xenon gas to enhance outcomes subsequent to surgical manipulation.

### CROSS-REFERENCE TO RELATED APPLICATIONS

This application claims priority to U.S. Provisional Application No. 62/436,408, filed Dec. 19, 2016, which is incorporated herein by reference in its entirety

### FIELD OF THE INVENTION

The invention pertains to the area of surgical arts, more specifically, the invention pertains to the field of minimally invasive surgery, more specifically, the invention pertains to the use of various gas mixtures in order to improve recovery time, decrease inflammation and decrease complications following laparoscopic surgery through the manipulation of gases used in insufflation.

### BACKGROUND OF THE INVENTION

Laparoscopic surgery, also called minimally or less invasive surgery (MIS or LIS) or keyhole surgery is a modern surgical technique in which operations in the body are performed through small incisions as compared to the larger incisions needed in traditional surgical procedures. Gas such as carbon dioxide is delivered, via an insufflator, into a body cavity such as the abdomen leading to the formation of a pneumoperitoneum, thereby providing sufficient space for the surgeon to operate. The insufflator maintains the pneumoperitoneum and acts to renew the gas when leaks occur. Gas such as carbon dioxide that is used for insufflation is both cold and dry and it is not surprising therefore those patients undergoing laparoscopic procedures often suffer a significant drop in core body temperature, which can result in considerable post-surgical pain and significant complications, such as cardiac stress, immunological and clotting problems, for the patient. Additionally, gases such as carbon dioxide do not possess ability to modify biological processes.

### DESCRIPTION OF THE INVENTION

The invention teaches the use of noble gases for insufflation during laparoscopic surgical procedures. Specifically, the invention teaches the previously unrecognized application of noble gases to prevent inflammation, accelerate wound healing, and suppress mediators of tissue damage such as the TNF-alpha, IL-1 beta, and IL-6 cytokines. Specifically, the invention further provides a means to prevent surgical adhesions from forming.

For the practice of the invention, it is important for the practitioner to understand some of the basic physiology of the peritoneal cavity, and particularly the serous membrane which comprises the lining of the abdominal cavity and it covers most of the intra-abdominal organs, which is termed the “peritoneum”. This layer is made of a single lining of mesothelium, generally 2.5-3 μm thick, supported by a thin layer of connective tissue [1]. On average the adult peritoneum possesses an area of approximately 4,000 cm² in adults [2], with its main physiological function being reduction of friction among abdominal viscera, and thus supporting their movement [3]. It also walls off infection and serves as a reservoir of fat, especially in the omentum. It is known that the peritoneum contains two distinct layers of collagen, and it is one of the most richly vascularised of all organs. The membrane comprises very large amounts of mucopolysaccharides or glycosaminoglycans and just beneath its surface there is an elastin layer that gives the peritoneum mobility [4]. The surface lining of the peritoneum consists of highly differentiated mesothelial cells [4, 5]. These cells are predominantly flattened, squamous-like, approximately 25 μm in diameter, with the cytoplasm raised over a central round or oval nucleus [6]. The mesothelial cells which comprise the peritoneum have well-developed cell-to-cell junctional complexes including tight junctions, adherent junctions, gap junctions and desmosomes. Tight junctions, in particular, are crucial for the development of cell surface polarity and the establishment and maintenance of a semi-permeable diffusion barrier. Furthermore, these cells produce into secretion glycosaminoglycans, proteoglycans and phospholipids to provide a slippery, non-adhesive glycocalyx that protects the serosal surface from abrasion, infection and tumour dissemination [7-9]. In addition, mesothelial cells generate cytokines, chemokines, growth factors and matrix components that regulate inflammation, initiate cell proliferation, differentiation and migration, and mediate tissue repair [10]. Providing scaffolding for the mesothelial cells are connective tissue proteins, and abundant vascular channels deliver oxygen and other nutrients to them. Interspersed among the connective tissue, there are extremely poorly differentiated and epithelioid-like cells similar to fibroblasts. These cells can undergo a variety of differentiation changes after exposure to injury or other types of stimuli, perhaps becoming mesothelial cells during peritoneal repair.

In contrast to standard surgery, laparoscopic surgery induces less direct trauma because of gentle tissue handling, haemostasis, and constant irrigation, the use of microsurgical instruments and the smaller operative field. This procedure has been associated with less postoperative pain, less systemic immunological depression, less wound infection, fewer complications, faster bowel recovery, shorter hospital stays and earlier return to normal activities; however, the operating times can be longer in comparison to those in open surgery. Typically, during laparoscopic surgery, the abdominopelvic cavity is first inflated with a gas to provide a space for viewing the surgical site and manipulating instruments. CO₂ is used almost universally as the insufflation agent to create this space called the laparoscopic pneumoperitoneum. CO₂ is the most common gas used for insufflation because of safety and supply reasons. First, it is non-combustible, eliminating the risk of fire when electro-surgical instruments are used, and second, it is cheap and highly soluble in water. Solubility is important as any gas trapped in the body following surgery must be removed. CO₂ dissolves into the serous fluid then migrates into the bloodstream where it travels to the lungs and is breathed out; therefore, CO₂ can easily be removed from the body without any major effect on the body's metabolism. This high solubility in water reduces the risk of gas embolism impairing cardiac function. In one embodiment of the invention, the high solubility of xenon in blood is leveraged so as to provide an alternative means of insufflating the peritoneum, while possessing anti-inflammatory, anti-fibrotic, and acceleration of wound healing.

One of the major causes of adhesion formation and injury during laparascopic surgery is the damage to the serous membrane that covers the abdominal cavity and most of the intra-abdominal organs. It is a very delicate layer highly susceptible to damage and it is not designed to cope with variable conditions associated with artificial inflation of the peritoneum.

Accordingly, the invention provides means of administering noble gases as a method of insufflation. In one embodiment a mixture of xenon and carbon dioxide gas is utilized to provide insufflation. Of the noble gases, xenon is preferred due to its highest solubility in water, plasma, and blood as compared to other noble gases.

Various aspects of the invention relating to the above are enumerated in the following paragraphs:

Aspect 1. A method of decreasing complications subsequent to manipulation of a tissue, said method comprising the steps of: a) providing a mixture containing one or more noble gases; b) administering said mixture containing one or more noble gases to the periphery of the area, or directly to the area to which said manipulation of tissue is to be performed; and c) optionally providing a means to perform extended release administration of said mixture containing one or more noble gases.

Aspect 2. The method of aspect 1, wherein said decreasing complications refers to acceleration of healing as compared to a subject undergoing the same manipulation without a therapeutic intervention by administration of said mixture containing one or more noble gases.

Aspect 3. The method of aspect 1, wherein said complications refers to fibrosis.

Aspect 4. The method of aspect 1, wherein said complications refers to formation of adhesions.

Aspect 5. The method of aspect 1, wherein said complications refers to pain.

Aspect 6. The method of aspect 1, wherein said complications refers to inflammation.

Aspect 7. The method of aspect 6, wherein said inflammation refers to enhanced TNF-alpha production.

Aspect 8. The method of aspect 6, wherein said inflammation refers to enhanced IL-6 production.

Aspect 9. The method of aspect 6, wherein said inflammation refers to enhanced IL-1 beta production.

Aspect 10. The method of aspect 6, wherein said inflammation refers to enhanced HMBG-1 production.

Aspect 11. The method of aspect 6, wherein said inflammation refers to enhanced fibrinogen production.

Aspect 12. The method of aspect 1, wherein said manipulation of a tissue is laparoscopic surgery.

Aspect 13. The method of aspect 1, wherein said manipulation of a tissue is surgery.

Aspect 14. The method of aspect 1, wherein said manipulation of a tissue is administration of a cellular therapy.

Aspect 15. The method of aspect 1, wherein said complication refers to death of cells comprising the peritoneum.

Aspect 16. The method of aspect 15, wherein said death of cells comprising the peritoneum is by means of necrosis.

Aspect 17. The method of aspect 15, wherein said death of cells comprising the peritoneum is by means of apoptosis.

Aspect 18. The method of aspect 15, wherein said death of cells comprising the peritoneum is by means of autophagy.

Aspect 19. The method of aspect 15, wherein said cells comprising the peritoneum are mesothelial cells.

Aspect 20. The method of aspect 15, wherein said cells comprising the peritoneum are connective tissue cells.

Aspect 21. The method of aspect 1, wherein said complications refers to increased friction among abdominal viscera.

Aspect 22. The method of aspect 1, wherein said noble gas is xenon.

Aspect 23. The method of aspect 22, wherein said noble gas is a mixture, said mixture containing one or more of the following gases: a) nitrogen; b) helium; c) Nitric Oxide; d) krypton; e) argon; and f) neon.

Aspect 24. The method of aspect 1, wherein said patient is treated with said noble gas containing mixture by incubating cells of the patient in an atmosphere containing said gases.

Aspect 25. The method of aspect 24, wherein said atmosphere is created by means of a sealed enclosure.

Aspect 26. The method of aspect 25, wherein said sealed enclosure is an incubator.

Aspect 27. The method of aspect 1, wherein said noble gas mixture containing said Noble gas is administered into said patient by dissolving of said noble gas or mixture containing said Noble gas into a liquid.

Aspect 28. The method of aspect 1, wherein said noble gas containing mixture is comprised of a gas mixture containing oxygen and a proportion by volume of 20 to 70% of xenon.

Aspect 29. The method of aspect 28, wherein said proportion of xenon is between 22 and 60% by volume to oxygen.

Aspect 30. The method of aspect 28, wherein said proportion of xenon is between 25 and 60% by volume to oxygen.

Aspect 31. The method of aspect 1, wherein said noble gas containing mixture consists only of a) oxygen and xenon or b) air and xenon.

Aspect 32. The method of aspect 1, wherein said noble gas containing mixture also contains nitrogen, helium, Nitric Oxide, krypton, argon or neon.

Aspect 33. The method of aspect 1, wherein said noble gas containing mixture contains a proportion by volume of oxygen of between 15 and 25%.

Aspect 34. The method of aspect 1, wherein said noble gas containing mixture is supplied for inhalation from a pressurized container at a pressure greater than 2 bar.

Aspect 35. The method of aspect 1, wherein said noble gas containing mixture is administered intranasally.

Aspect 36. The method of aspect 1, wherein said noble gas containing mixture is administered through the use of a hyperbaric chamber.

Aspect 37. The method of aspect 36, wherein said hyperbaric chamber is pressurized to a pressure of no more than 3 atm (0.3 MPa).

Aspect 38. The method of aspect 36, wherein a noble gas is administered to the patient while the patient is in the hyperbaric environment.

Aspect 39. The method of aspect 1 wherein said noble gas is administered by inhalation or simulated inhalation.

Aspect 40. The method of aspect 1, wherein said noble gas is xenon, helium, or a mixture of xenon and helium.

Aspect 41. The method of aspect 1, wherein the noble gas is xenon or a mixture of xenon and helium, and the partial pressure of xenon is no more than about 0.8 atm (0.08 MPa).

Aspect 42. The method of aspect 1, wherein said noble gas is administered mixed with air, the air partial pressure being about 1 atm (0.1 MPa).

Aspect 43. The method of aspect 1, wherein said noble gas is administered as part of a gas mixture comprising oxygen, the nitrogen partial pressure in the mixture being equal to or less than about 0.8 atm (0.08 MPa).

Aspect 44. The method of aspect 43, wherein said gas mixture is essentially free of nitrogen.

Aspect 45. The method of aspect 43, wherein the oxygen partial pressure is about 0.2 atm (0.02 MPa).

Aspect 46. A method of decreasing inflammation in a laparoscopic surgical procedure: a) providing a mixture containing one or more noble gases; b) administering said mixture containing one or more noble gases as part of a gas used to perform insufflation of the peritoneum; and c) optionally providing a means to perform extended release administration of said mixture containing one or more noble gases.

Aspect 47. The method of claim 46, wherein said noble gas is selected from a group comprising of: a) helium; b) neon; c) argon; d) xenon; and e) krypton.

Aspect 48. The method of aspect 47, wherein said noble gas is argon, administered at a higher than atmospheric pressure.

Aspect 49. The method of aspect 47, wherein said noble gas is neon, administered at a higher than atmospheric pressure.

Aspect 50. The method of aspect 47, wherein said noble gas is krypton, administered at a higher than atmospheric pressure.

Aspect 51. The method of aspect 47, wherein said noble gas is helium, administered at a higher than atmospheric pressure.

Aspect 52. The method of aspect 47, wherein said noble gas is helium, administered at a higher than atmospheric pressure.

Aspect 53. The method of aspect 47, wherein said composition comprises 5-40% xenon gas.

Aspect 54. The method of aspect 47, wherein said xenon gas is admixed with air

Aspect 55. The method of aspect 47, wherein said xenon gas is admixed with carbon dioxide.

Aspect 56. The method of aspect 47, wherein said xenon gas is admixed with an oxygen/nitrogen mixture.

Aspect 57. The method of aspect 1, wherein xenon gas is used at concentrations up to 100% as a means of insufflation.

Aspect 58. The method of aspect 1, wherein helium gas is used at concentrations up to 100% as a means of insufflation.

Aspect 59. The method of aspect 1, wherein neon gas is used at concentrations up to 100% as a means of insufflation.

Aspect 60. The method of aspect 1, wherein argon gas is used at concentrations up to 100% as a means of insufflation.

Aspect 61. The method of aspect 1, wherein krypton gas is used at concentrations up to 100% as a means of insufflation.

Aspect 62. The method of aspect 57, wherein carbon dioxide gas is used to dilute xenon as a means of insufflation.

Aspect 63. The method of aspect 57, wherein carbon dioxide gas is used to dilute helium as a means of insufflation.

Aspect 64. The method of aspect 57, wherein carbon dioxide gas is used to dilute neon as a means of insufflation.

Aspect 65. The method of aspect 57, wherein carbon dioxide gas is used to dilute argon as a means of insufflation.

Aspect 66. The method of aspect 57, wherein carbon dioxide gas is used to dilute krypton as a means of insufflation.

Aspect 67. The method of aspect 1, wherein said gas used for insufflation is humidified by means of addition of water or a water containing fluid.

Aspect 68. The method of aspect 47, wherein said gas used for insufflation is humidified by means of addition of water or a water containing fluid.

Aspect 69. The method of aspect 1, wherein said noble gas is introduced as a dissolved gas in a liquid means serving as a humidifying agent.

Aspect 70. A composition containing a noble gas that is used for washing the surgical area in a manner to prevent post-surgical complications.

### REFERENCES

1. Slater, N. J., A. T. Raftery, and G. H. Cope, The ultrastructure of human abdominal mesothelium. J Anat, 1989. 167: p. 47-56.
2. Albanese, A. M., et al., Peritoneal surface area: measurements of 40 structures covered by peritoneum: correlation between total peritoneal surface area and the surface calculated by formulas. Surg Radiol Anat, 2009. 31(5): p. 369-77.
3. Coakley, F. V. and H. Hricak, Imaging of peritoneal and mesenteric disease: key concepts for the clinical radiologist. Clin Radiol, 1999. 54(9): p. 563-74.
4. Suzuki, T., et al., An injured tissue affects the opposite intact peritoneum during postoperative adhesion formation. Sci Rep, 2015. 5: p. 7668.
5. Mutsaers, S. E., The mesothelial cell. Int J Biochem Cell Biol, 2004. 36(1): p. 9-16.
6. Henderson, D. W., K. B. Shilkin, and D. Whitaker, Reactive mesothelial hyperplasia vs mesothelioma, including mesothelioma in situ: a brief review. Am J Clin Pathol, 1998. 110(3): p. 397-404.
7. Bodega, F., et al., Lubricating recovery of damaged pleural mesothelium: effect of time and of phosphatidylcholines. Respir Physiol Neurobiol, 2014. 203: p. 116-20.
8. Mutsaers, S. E., D. Whitaker, and J. M. Papadimitriou, Changes in the concentration of microvilli on the free surface of healing mesothelium are associated with alterations in surface membrane charge. J Pathol, 1996. 180(3): p. 333-9.
9. Yung, S. and T. M. Chan, Hyaluronan—regulator and initiator of peritoneal inflammation and remodeling. Int J Artif Organs, 2007. 30(6): p. 477-83.
10. Yung, S. and T. M. Chan, Mesothelial cells. Perit Dial Int, 2007. 27 Suppl 2: p. S110-5.

1. A method of decreasing complications subsequent to manipulation of a tissue, said method comprising the steps of: a) providing a mixture containing one or more noble gases; b) administering said mixture containing one or more noble gases to the periphery of the area, or directly to the area to which said manipulation of tissue is to be performed; and c) optionally providing a means to perform extended release administration of said mixture containing one or more noble gases.

2. The method of claim 1, wherein said decreasing complications refers to acceleration of healing as compared to a subject undergoing the same manipulation without a therapeutic intervention by administration of said mixture containing one or more noble gases.

3. The method of claim 1, wherein said manipulation of a tissue is laparoscopic surgery.

4. The method of claim 1, wherein said complication refers to death of cells comprising the peritoneum.

5. The method of claim 4, wherein said death of cells comprising the peritoneum is by means of necrosis.

6. The method of claim 4, wherein said death of cells comprising the peritoneum is by means of apoptosis.

7. The method of claim 1, wherein said noble gas is xenon.

8. The method of claim 7, wherein said noble gas is a mixture, said mixture containing one or more of the following gases: a) nitrogen; b) helium; c) Nitric Oxide; d) krypton; e) argon; and f) neon.

9. The method of claim 1, wherein said patient is treated with said noble gas containing mixture by incubating cells of the patient in an atmosphere containing said gases.

10. The method of claim 9, wherein said atmosphere is created by means of a sealed enclosure.

11. The method of claim 10, wherein said sealed enclosure is an incubator.

12. The method of claim 1, wherein said noble gas mixture containing said Noble gas is administered into said patient by dissolving of said noble gas or mixture containing said Noble gas into a liquid.

13. The method of claim 1, wherein said noble gas containing mixture is comprised of a gas mixture containing oxygen and a proportion by volume of 20 to 70% of xenon.

14. The method of claim 13, wherein said proportion of xenon is between 22 and 60% by volume to oxygen.

15. The method of claim 13, wherein said proportion of xenon is between 25 and 60% by volume to oxygen.

16. The method of claim 1, wherein said noble gas containing mixture consists only of a) oxygen and xenon or b) air and xenon.

17. The method of claim 1, wherein said noble gas containing mixture also contains nitrogen, helium, Nitric Oxide, krypton, argon or neon.

18. The method of claim 1, wherein said noble gas containing mixture contains a proportion by volume of oxygen of between 15 and 25%.

19. The method of claim 1, wherein said noble gas containing mixture is supplied for inhalation from a pressurized container at a pressure greater than 2 bar.

20. A method of decreasing inflammation in a laparoscopic surgical procedure: a) providing a mixture containing one or more noble gases; b) administering said mixture containing one or more noble gases as part of a gas used to perform insufflation of the peritoneum; and c) optionally providing a means to perform extended release administration of said mixture containing one or more noble gases.
